(19) United States
(12) Patent Application Publication
(10) Pub. No.: US 2002/0192272 A1
(43) Pub. Date: Dec. 19, 2002

(54) Metronidazole pledgets

(76) Inventor: Karl F. Popp, Schodack Landing, NY (US)

Correspondence Address: LOTT & FRIEDLAND, P.A., P.O. BOX 141098, CORAL GABLES, FL, 33114-1098, US

(21) Appl. No.: 10/044,275

(22) Filed: Jan. 10, 2002

Related U.S. Application Data
(60) Provisional application No. 60/279,382, filed on Mar. 28, 2001.

(51) Int. Cl. A61K 31/4168; A61K 9/70

(52) U.S. Cl. 424/449; 442/123; 514/398

(57) ABSTRACT

A dermatological delivery system comprising a topically acceptable, inert support selected from the group consisting of cotton, rayon, polyester, polypropylene, wood pulp, mohair, nylon fleece, and neoprene foam, or combination thereof, impregnated with an about 0.1% to about 2% solution of metronidazole; the solution having a major solvent component comprising water, ethanol or a mixture of water and ethanol, the support being operable to permit application of the solution to the skin.

### CLAIM OF PRIORITY

This application is related to provisional application Serial No. 60/279,382 filed on Mar. 28, 2001 based upon which priority is claimed pursuant to 35 U.S.C. § 119(e).

### TECHNICAL FIELD

The present invention relates to a novel method of treatment of skin disorders using metronidazole pledgets.

### BACKGROUND OF THE INVENTION

Metronidazole, (2-Methyl-5-nitroimidazole-1-ethanol), has an extremely broad spectrum of protozoal and antimicrobial activity. Metronidazole is clinically effective in trichomoniasis, amebias, and giardiasis, as well as in a variety of infectious caused by obligate, anaerobic, bacteria, including Bacteroides fragilis. Metronidazole is clinically administered both orally and intravenously. Metronidazole has also been reported to be effective via both oral and topically application in the treatment of skin disorders including acne, impetigo, and rosacea. See for example, Schirner, A., and Haneke, E., Rosacea and Metronidazole, Acta Dermatologica, 7,27-30 (1981); and Nielsen, P. G., A Double Blind Study for 1% Metronidazole Cream Reserves Systemic Oxytetracycline Therapy for Rosacea, British Journal of Dermatology, 109, 63-65, (1983).

Rosacea is an acne form condition primarily affecting the areas of the nose, cheeks, and forehead of adults. The condition is characterized by erythema, papules, rhinophyma, and telagiectases. The cause of rosacea is unknown; however; dietary influence, gastrointestinal disturbances, psychologic or hormonal imbalance, sebaceous gland abnormalities, and infection have been considered but not validated. Other theories range from solar-induced dermal connective tissue damage, with resultant vascular distension to humorally mediated active vasodilatory changes. A causative role has also been suggested for the hair follicle mite, Demodex, C. E. Bonnard, et al., The Demodex Mite Population, J. Amer. Acad. Dermatology, Vol. 28, No. 3, pp. 443-447, March 1993.

Missing in the art is a convenient means to ensure patient compliance with topical administration of a metronidazole solution. At present, there is no commercially available pledget form of metronidazole. Wang, et al. Degredation Kinetics of Metronidaxole in Solution, J. Pharm Sciences, 82 (1), pp 95-97 (January 1993) teaches that metronidazole degrades in solution.

The method of treatment of the present invention overcomes the failures of a single course therapy with a novel treatment using metronidazole in a convenient pledget form.

### SUMMARY OF THE INVENTION

In accordance with the present invention, the treatment of Rosacea and other acneform skin conditions is accomplished with topical metronidazole administered in a pledget form.

Numerous other advantages and features of the present invention will become readily apparent from the following description of the preferred embodiments of the invention, the accompanying examples, and the appended claims.

### DESCPIPTION OF THE PREFERRED EMBODIMENTS

The present invention pertains to a topically acceptable, inert support impregnated with a solution of antimicrobially effective concentration of metronidazole. The support carries the solution and is operable to permit its application to the skin. Typically, the support is a fiber matrix, that may be woven or non-woven, or a polymeric sponge. Typical support materials include cotton, rayon, polyester, polypropylene, wood pulp, mohair, nylon fleece, or neoprene foam.

The term “metronidazole” as used in this specification and claims is meant to include not only 2-methyl-5-nitrolmidazole-1-ethanol, but also those analogs and derivatives of metronidazole which are pharmaceutically desirable and which have therapeutic activity when orally administered and/or topically applied. Metronidazole is employed in the treatment in a therapeutically effective amount. The actual dosage or concentration of metronidazole may vary, depending upon the nature and degree of the disorders being treated, and whether the drug is being administered for therapeutic or prophylactic purposes.

When administered orally, the daily dose of metronidazole ranges from about 10 milligrams to about 2 grams, preferably from about 50 milligrams to 1000 milligrams. Topical compositions would comprise at least 0.1 wt %, up to 5 wt-%, preferably from about 0.25 to 3 wt %. Topical compositions are preferably delivered in a volume about 0.1 to about 10 ml and most preferably about 5 ml.

The solution has a major solvent component which comprises at least one member selected from the group consisting at topically acceptable liquid alkanols and water. The solution can also be a mixture of liquid alkanols and water. Preferably, the solvent is water, ethanol or a mixture of ethanol and water. Alkanols when used can be present in any amount. A preferred percentage of such alkanols is 5-95% and a most preferred percentage is 20-80%. Ethanol is the most preferred alkanol. Optionally, one or more polyols such as glycerine or propylene glycol can be added.

Examples of pharmaceutical dosage forms are demonstrated in the following examples. These examples are meant to illustrate the invention, the scope of the invention is limited only by the appended claims. Variations in the compositions which do not adversely affect the effectiveness of the antibiotics will be evident to one skilled in the art, and or within the scope of this invention. For example, additional ingredients, such as coloring agents, sunscreens, and the like, may be included in the compositions, as long as the resulting composition retains the desirable properties, e.g., non-comedogenicity, high specific activity, and the like, described above.

The products of the present invention comprise a water insoluble substrate. By “water insoluble” is meant that the substrate does not dissolve in or readily break apart upon immersion in water. The water insoluble substrate is the implement or vehicle for delivering the antimicrobial metronidazole composition of the present invention to the area to be treated.

Nonlimiting examples of suitable insoluble substrates which meet the above criteria include non-woven substrates, woven substrates, hydro-entangled substrates, air entangled substrates, synthetic sponges, polymeric netted meshes, and the like. By non-woven is meant that the layer is comprised of fibers which are not woven into a fabric but rather are formed into a sheet, mat, or pad layer. The fibers can either be random (i.e., randomly aligned) or they can be carded (i.e. combed to be oriented in primarily one direction). Furthermore, the non-woven substrate can be composed of a combination of layers of random and carded fibers.

Non-woven substrates may be comprised of a variety of materials both natural and synthetic. By natural is meant that the materials are derived from plants, animals, insects or by-products of plants, animals, and insects. By synthetic is meant that the materials are obtained primarily from various man-made materials or from natural materials that have been further altered. The conventional base starting material is usually a fibrous web comprising any of the common synthetic or natural textile-length fibers, or mixtures thereof.

Methods of making non-woven substrates are well known in the art. Generally, these non-woven substrates can be made by air-laying, water-laying, melt-blowing, co-forming, spun-bonding, or carding processes in which the fibers or filaments are first cut to desired lengths from long strands, passed into a water or air stream, and then deposited onto a screen through which the fiber-laden air or water is passed. The resulting layer, regardless of its method of production or composition, is then subjected to at least one of several types of bonding operations to anchor the individual fibers together to form a self-sustaining web. In the present invention the non-woven layer can be prepared by a variety of processes including hydro-entanglement, thermally bonding or thermo-bonding, and combinations of these processes. Moreover, the substrates of the present invention can consist of a single layer or multiple layers. In addition, a multi-layered substrate can include films and other non-fibrous materials.

The substrate can be made into a wide variety of shapes and forms including flat pads, thick pads, thin sheets, ball-shaped implements, irregularly shaped implements, and having sizes ranging from a surface area of about a square inch to about hundreds of square inches. The exact size will depend upon the desired use and product characteristics. Especially convenient are square, circular, rectangular, or oval pads having a surface area of from about 0.5 in² to about 144 in², preferably from about 1 in² to about 25 in², and more preferably from about 1 in² to about 4 in², and a thickness of from about 1 mil to about 500 mil, preferably from about 5 mil to about 250 mil, and more preferably from about 10 mil to about 100 mil.

The water insoluble substrates of the present invention can comprise two or more layers, each having different textures and abrasiveness. The differing textures can result from the use of different combinations of materials or from the use of different manufacturing processes or a combination thereof. A dual textured substrate can be made to provide the advantage of having a more abrasive side for exfoliation and a softer, absorbent side for gentle cleansing. In addition, separate layers of the substrate can be manufactured to have different colors, thereby helping the user to further distinguish the surfaces.

Non-limiting examples of materials useful for the substrate in the present invention include: cotton, rayon, polyester, wood pulp with latex binder, rayon/polyester, rayon/polypropylene, rayon/polypropylene/cotton or cotton/polyester. A preferred substrate is a combination of polyester and rayon. Most preferred is a substrate of 50% polyester and 50% rayon.

To protect stability, it is desirable to package the metronidazole pledget in a light and oxygen blocking barrier. Examples of suitable packaging materials include: Polyester/Polyethylene/Foil/Barex; Cellophane/Polyester/Foil/Co-extruded Polyethylene; Cellophane/Polyethylene/Foil/Poluethyne; Cellophane/Polyethylene/Foil/Surlyn; Polyester /Polyethylene/Foil/Sclair; Cellophane/Polyethylene/Foil/Foil/co-polymer Paper/Polyethylene/Foil/PET; (polyethyleneterephallate)/Polyethylene Paper/Polyethylene/Foil/Co-extruded Polyethylene; Polyester/Polyethylene/Foil/Ethylene Acrylic Acetate/Polyethylene; Polyester/Polyethylene/Foil/Ethylene Methyl Acrylate Polyethylene; PET/Polyethylene/Foil/Barex.

Stability can be further enhanced by introducing inert gas, including but not limited to argon or nitrogen, into the packaging.

Jars and bottles suitable for storage of the invention can be fabricated from conventional materials such as glass, polypropylene, polyethylene blends, polyethylene, polyethyleneterephthalate, and blends of polypropylene, polyethylene and polyethyleneterephthalate.

The product is applied by opening the container containing the pledget and applying the pledget to the desired areas of the skin. Pledget products can be packaged such that each container only has a single pledget or the containers can contain multiple pledgets.

An alternative delivery system employs a “dab-o-matic” type package delivery system. Such systems include a storage means containing the solution of active agent, a pad type applicator for delivery of the active agent from the bottle to the skin, and a cap. The storage means can optionally be pressurized using aerosol technology for convenience or more careful dosing of the active agent.

The patient uses the dab-o-matic type device by removing the cap and applying the product by contacting the applicator to the skin. A spring mechanism opens a valve allowing the solution of active agent to flow through the applicator to the skin.

Representative formulations suitable for impregnation onto pledgets can be made in accordance with Examples 1, 2, 3 or 4 set forth below.

| | |
|---|---|
| COMPONENT | w/w % |

EXAMPLE 1

| | |
|---|---|
| Metronidazole | 0.75 |
| Disodium EDTA | 0.10 |
| Propylene Gycol | 3.00 |
| Water | 96.16 |
| To Make Total | 100.00 |

EXAMPLE 2

| | |
|---|---|
| Metronidazole | 2.00 |
| Glycerin | 5.00 |
| Alcohol | 93.00 |
| To Make Total | 100.00 |

EXAMPLE 3

| | |
|---|---|
| Metronidazole | 1.25 |
| Glycerin | 10.10 |
| Alcohol | 20.00 |
| Water | 68.75 |
| To Make Total | 100.00 |

EXAMPLE 4

| | |
|---|---|
| Metronidazole | 0.760 |
| Alcohol SDA 40 B | 17.5 |
| Glycerin, USP | 10.0 |
| Sodium Phosphate, | 0.200 |
| Monobasic | |
| Benzyl Alcohol, NF | 2.50 |
| Water | 69.04 |
| To Make Total | 100.00 |

Each of the formulations in the above examples are created by mixing all components in a suitable container.

The Formulation of Example 4 was impregnated on a white 50% polyester/50% rayon pad and placed on stability. Table 1 shows that there was minimal degradation of metronidazole during the stability testing

TABLE 1
Stability
METRONIZADOLE (% w/v)

| Storage | # of Days | Result | % Label |
|---|---|---|---|
| 25/60 | 30 | 0.735 | 98.0 |
| 25/60 | 60 | 0.732 | 97.6 |
| 25/60 | 91 | 0.744 | 99.2 |
| 30/60 | 60 | 0.736 | 98.1 |
| 30/60 | 91 | 0.742 | 98.9 |
| 40/75 | 30 | 0.744 | 99.2 |
| 40/75 | 60 | 0.738 | 98.4 |
| 40/75 | 91 | 0.739 | 98.5 |
| 6-1 | 30 | 0.746 | 99.5 |
| 6-1 | 60 | 0.737 | 98.3 |

TABLE 1
Stability
METRONIZADOLE (% w/v)

| Storage | # of Days | Result | % Label |
|---|---|---|---|
| 6-1 | 91 | 0.742 | 98.9 |
| FT | 91 | 0.740 | 98.7 |

Table 2 shows that there was minimal degradation of the above formulation 1 into unknown related substances:

TABLE 2
Stability
METRONIZADOLE (% w/v) Unknown Related Substances

| Storage | # of Days | Result | % Label |
|---|---|---|---|
| 25/60 | 30 | 0.1 | na |
| 25/60 | 60 | 0.1 | na |
| 25/60 | 91 | 0.1 | na |
| 30/60 | 60 | 0.1 | na |
| 30/60 | 91 | 0.1 | na |
| 40/75 | 30 | 0.1 | na |
| 40/75 | 60 | 0.1 | na |
| 40/75 | 91 | 0.1 | na |
| 6-1 | 30 | 0.1 | na |
| 6-1 | 60 | 0.1 | na |
| 6-1 | 91 | 0.1 | na |
| FT | 91 | 0.1 | na |

Table 3 sets out the result of analysis for the 4-nitro degradation isomer of metronidazole.

TABLE 3
METRONIZADOLE (% w/v) 4-nitro-isomer

| Storage | # of Days | Result | % Label |
|---|---|---|---|
| 25/60 | 30 | 0.1 | na |
| 25/60 | 60 | 0.1 | na |
| 25/60 | 91 | 0.1 | na |
| 30/60 | 60 | 0.1 | na |
| 30/60 | 91 | 0.1 | na |
| 40/75 | 30 | 0.1 | na |
| 40/75 | 60 | 0.1 | na |
| 40/75 | 91 | 0.1 | na |

TABLE 3
METRONIZADOLE (% w/v) 4-nitro-isomer

| Storage | # of Days | Result | % Label |
|---|---|---|---|
| 6-1 | 30 | 0.1 | na |
| 6-1 | 60 | 0.1 | na |
| 6-1 | 91 | 0.1 | na |
| FT | 91 | 0.1 | na |

Table 4 sets out the result of analysis for the 2-methyl-5-nitroimidazole degredation product of metronidazole.

TABLE 4
Stability
METRONIZADOLE (% w/v) 2-methyl-5-nitroimidazole

| Storage | # of Days | Result | % Label |
|---|---|---|---|
| 25/60 | 30 | 0.1 | na |
| 25/60 | 60 | 0.1 | na |
| 25/60 | 91 | 0.1 | na |
| 30/60 | 60 | 0.1 | na |
| 30/60 | 91 | 0.1 | na |
| 40/75 | 30 | 0.1 | na |
| 40/75 | 60 | 0.1 | na |
| 40/75 | 91 | 0.1 | na |
| 6-1 | 30 | 0.1 | na |
| 6-1 | 60 | 0.1 | na |
| 6-1 | 91 | 0.1 | na |
| FT | 91 | 0.1 | na |

Table 5 sets out the result of analysis for pH of samples placed on stability.

TABLE 5
Stability
METRONIZADOLE Pledget pH

| Storage | # of Days | Result | % Label |
|---|---|---|---|
| 25/60 | 30 | 6.6 | na |
| 25/60 | 60 | 6.4 | na |
| 30/60 | 60 | 6.7 | na |
| 30/60 | 91 | 6.5 | na |
| 40/75 | 30 | 6.9 | na |

TABLE 5
Stability
METRONIZADOLE Pledget pH

| Storage | # of Days | Result | % Label |
|---|---|---|---|
| 40/75 | 91 | 6.8 | na |
| 6-1 | 60 | 6.5 | na |
| 6-1 | 91 | 6.3 | na |
| FT | 91 | 6.5 | na |

These data support that stable metronidazole pledget products can be developed using the invention disclosed herein.

What is claimed is:

1. A dermatological delivery system comprising a topically acceptable, inert support impregnated with a metronidazole solution, said metronidazole solution including a major solvent component.

2. A dermatological delivery system according to claim 1 wherein said support is a woven fiber matrix.

3. A dermatological delivery system according to claim 1 wherein said support is a non-woven fiber matrix.

4. A dermatological delivery system according to claim 1 wherein said support is a polymeric sponge.

5. A dermatological delivery system according to claim I wherein said support is selected from the group consisting of cotton, rayon, polyester, polypropylene, wood pulp, mohair, nylon fleece and neoprene foam, and a combination thereof.

6. The delivery system according to claim 1 wherein said support is rayon and polyester.

7. The delivery system according to claim 1 wherein the support comprises from 20%-80% rayon and from 20%-80% polyester.

8. The delivery system according to claim 1 wherein the support system is 50% polyester and 50% rayon.

9. A dermatological delivery system according to claim 1 wherein said major solvent component is ethanol.

10. The delivery system according to claim 9 wherein the ethanol is present in an amount between 0%-100%.

11. The delivery system according to claim 9 wherein the ethanol is present in an amount of between 50%-80%

12. A dermatological delivery system according to claim 1 wherein said major solvent component is water.

13. The delivery system according to claim 12 wherein the water is present in an amount between 0%-100%.

14. A dermatological delivery system according to claim 1 wherein said major solvent component is a mixture of water and ethanol.

15. A dermatological delivery system according to claim 1 wherein said major solvent component comprises water, ethanol or a mixture of water and ethanol, and at least one polyol.

16. A dermatological delivery system according to claim 1 wherein the metronidazole is present in a concentration of from about 0.1% to about 2%.

17. The delivery system according to claim 1 wherein the metronidazole is present in a concentration of 0.75%.

18. The delivery system according to claim 1 wherein the metronidazole is present in a concentration of 1.25%.

19. The delivery system according to claim 1 wherein the metronidazole is present in a concentration of 2.0%.

20. The delivery system of claim 1 wherein the volume of said metronidazole solution delivered is from about 0.1 to about 10 ml.

21. The delivery system of claim 1 wherein the volume of said metronidazole solution delivered is about 5 ml.

22. The delivery system of claim 1 wherein the inert support is from about 0.5 in² to about 144 in² in area.

23. The delivery system of claim 1 wherein the inert support is from about 1 in² to about 4 in² in area.

24. The delivery system of claim 1 wherein the inert support is from about 1 mil to about 500 mils thick.

25. The delivery system of claim 1 wherein the inert support is from about 5 mils to about 250 mils thick.

26. The delivery system of claim 1 wherein the inert support is from about 10 mils to about 100 mils thick.

27. A dermatological delivery system comprising a topically acceptable, inert support selected from the group consisting of cotton, rayon, polyester, polypropylene, wood pulp, mohair, nylon fleece, and neoprene foam, or combination thereof, impregnated with an about 0.1% to about 2% solution of metronidazole; said solution having a major solvent component comprising water, ethanol or a mixture of water and ethanol, said support being operable to permit application of said solution to the skin.

28. A dermatological delivery system comprising an alcoholic or aqueous solution of metronidazole in an antimicrobially effective concentration impregnated on a topically acceptable, inert support which is a woven or non-woven fiber matrix or a polymeric sponge.

29. The delivery system of claim 1 wherein the inert support is single use.

30. The delivery system of claim 1 wherein the inert support is part of a multiple dosing device having a storage means for multiple doses of metronidazole.

31. The delivery system of claim 30 wherein the multiple dosing device contains from 1-250 ml of metronidazole solution.

32. The delivery system of claim 30 wherein the multiple dosing device is a dab-o-matic.

33. The delivery system of claim 30 wherein the storage means comprises plastic, glass or metal.

34. The delivery system of claim 30 wherein the storage means comprises one or more of the following: polyester, polypropylene, polyethylene, glass, steel or aluminum.

35. The delivery system of claim 30 wherein the multiple dosing device is pressurized.

36. A dermatological delivery system as in claim 1 in which the delivery system is packaged in a light and/or oxygen blocking barrier.

37. A dermatological delivery system as in claim 36 in which the blocking barrier is selected from at least one of the following: Polyester/Polyethylene/Foil/Barex; Cellophane/Polyester/Foil/Co-extruded Polyethylene; Cellophane/Poly-ethylene/Foil/Poluethyne; Ce-llophane/Polyethylene/Foil/Surlyn;Polyester/Polyethylene/Foil/Sclair; Cellophane/Polyethylene/Foil/Foil/co-polymer Paper/Polyethylene/Foil/PET; (polyethyleneterephallate)/Polyethylene Paper/Polyethylene/Foil/Co-extruded Polyethylene; Polyester/Polyethylene/Foil/Ethylene Acrylic Acetate/Polyethylene; Polyester/Polyethylene/Foil/Ethylene Methyl Acrylate Polyethylene; PET/Polyethylene/Foil/Barex.

38. A method of treating dermatological conditions comprising topical administration of an effective amount of metronidazole using a delivery system comprising a topically acceptable, inert support impregnated with a metronidazole solution, said metronidazole solution including a major solvent component.

39. The method of claim 38 wherein the dermatological condition is any condition suitable for treatment with topical metronidazole.

40. The method of claim 38 wherein the dermatological condition is rosacea.

41. The method of claim 38 wherein the dermatological condition is acne.

42. The method of claim 38 wherein the dermatological condition is a metronidazole susceptible infection.

43. The delivery system of claim 1 having a metronidazole degradent content of less that 0.1%

44. The delivery system of claim 43 wherein 2 methyl 5-nitrometronidazole is present at less than 1%.

45. The delivery system of claim 43 wherein metronidazole 4-nitro isomer is present at less than 1%.
